(19) United States
(12) Patent Application Publication
(10) Pub. No.: US 2011/0318770 A1
(43) Pub. Date: Dec. 29, 2011

(54) COMPOSITIONS, KITS AND METHODS FOR DETERMINING PLASMIN ACTIVITY

(75) Inventors: Kevin Yarbrough, Apex, NC (US); Maria Cruz, Cary, NC (US); Pete Vandeberg, Apex, NC (US)

(73) Assignee: Talecris Biotherapeutics, Inc., Research Triangle Park, NC (US)

(21) Appl. No.: 13/145,875

(22) PCT Filed: Feb. 5, 2010
(86) PCT No.: PCT/US10/23296

§ 371 Date: Sep. 14, 2011

Related U.S. Application Data
(60) Provisional application No. 61/150,428, filed on Feb. 6, 2009.

(51) Int. Cl. C12Q 1/37; C07K 5/093

(52) U.S. Cl. 435/23; 530/331

(57) ABSTRACT

Compositions, kits, and methods for determining plasmin activity using a fluorogenic peptide substrate are provided.

### FIELD OF THE INVENTION

The present invention relates to compositions, kits, and methods for determining plasmin activity, in particular for determining plasmin activity using a fluorogenic peptide substrate, in particular a coumarin-peptide substrate.

### BACKGROUND OF THE INVENTION

A chromogenic substrate for plasmin activity is the peptide based substrate known as S-2403 (pyro-glu-phe-lys-p-nitroanilide). It has been shown that plasmin cuts the bond after the lysine residue of S2403 to release p-nitroanaline and a measure of p-nitroanaline has been shown to be directly related to plasmin activity. However, prior compositions and methods for determining plasmin activity have limits including specificity and sensitivity of detection.

There remains a need for effective compositions, kits, and methods for determining plasmin activity.

### SUMMARY OF THE INVENTION

There is now provided a substrate useful for determining plasmin activity.

In one aspect, the present invention provides a compound of the formula:

Rₘ-[Aₙ³A²A¹]-X (I)

wherein

A¹ is Lys; A² is Phe; A³ is an amino acid residue;

R is pyroglutamate or an N-terminal protecting group;

X is a fluorogenic group;

m is 0 or 1; and

n is an integer ≧0.

In another aspect, the present invention provides a method for determining an activity of a plasmin, the method comprising:

contacting the plasmin with a compound of the formula:

Rₘ-[Aₙ³A²A¹]-X (I)

wherein

A¹ is Lys; A² is Phe; A³ is an amino acid residue;

R is pyroglutamate or an N-terminal protecting group;

X is a fluorogenic group;

m is 0 or 1; and

n is an integer ≧0.

In some aspects, the present invention provides a method of determining pharmacokinetic of a plasmin, the method comprising:

contacting the plasmin with a compound of the formula:

Rₘ-[Aₙ³A²A¹]-X (I)

wherein

A¹ is Lys; A² is Phe; A³ is an amino acid residue;

R is pyroglutamate or an N-terminal protecting group;

X is a fluorogenic group;

m is 0 or 1; and

n is an integer ≧0, wherein the plasmin is obtained from a biological sample from a subject administered with the plasmin.

In other aspects, the present invention provides a method for determining a molecule that affects plasmin activity, the method comprising:

contacting the molecule with the plasmin in the presence of a compound of the formula:

Rₘ-[Aₙ³A²A¹]-X (I)

wherein

A¹ is Lys; A² is Phe; A³ is an amino acid residue;

R is pyroglutamate or an N-terminal protecting group;

X is a fluorogenic group;

m is 0 or 1; and

n is an integer ≧0.

Also provided are kits comprising one or more of the compounds of the present invention.

### BRIEF DESCRIPTION OF THE DRAWINGS

FIG. 1 is a graph showing relative responses with three different fluorogenic substrates: (a) Tal-TBMW2, which corresponds to one embodiment of a compound of the present invention and which can be represented by the formula: pyroglutamate-Phe-Lys-7-amido-4-methylcoumarin; (b) Sigma V-3138 (Sigma Aldrich, St. Louis, Mo.); and (c) S2403 (Chromogenix, Milano, Italy).

### DETAILED DESCRIPTION

### I. Composition

In one aspect, the present invention provides a compound of the formula:

Rₘ-[Aₙ³A²A¹]-X (I)

wherein

A¹ is Lys; A² is Phe; A³ is an amino acid residue;

R is pyroglutamate or an N-terminal protecting group;

X is a fluorogenic group;

m is 0 or 1; and

n is an integer ≧0.

Non-limiting examples of fluorogenic groups include 4-(substituted) coumarin, coumarin derivatives, hydroxy allyloxypropyl napthalimide quat, 4-methoxy-N-(3-N′N′-dimethylaminopropyl) napthalimide, 2-hydroxy-3-allyloxypropyl quat, 8-(3-vinylbenzyloxy)-1,3,6-pyrene trisulfonic acid, 8-(4-vinylbenzyloxy)-1,3,6-pyrene trisulfonic acid, 8-(allyloxy)-1,3,6-pyrene trisulfonic acid, 1-(substituted) naphthalene, 9-(substituted) anthracene, 2-(substituted) quinoline monohydrochloride, 2-(substituted) benzimidazole, 5-(substituted) fluorescein, 3-(substituted)-6,7-dimethoxy-1-methyl-2(1H)-quinoxazolinone, mixtures thereof and derivatives thereof.

In one embodiment, the fluorogenic group is 7-amino-4-methylcoumarin.

R can be pyroglutamate or an N-terminal protecting group. The N-terminal protecting group can protect the N-terminus of the peptide segment of the compound of the present invention. Examples of an N-terminal protecting group include, but are not limited to, an acyl protecting group, an aromatic urethane protecting group, and an aliphatic urethane protecting group. For example, the acyl protecting group can be succinyl, methoxysuccinyl, acetyl, benzoyl, and trifluoroacetyl.

In one embodiment, R is pyroglutamate.

In another embodiment, R is an N-terminal protecting group. In some embodiments, the N-terminal protecting group is succinyl or methoxysuccinyl.

The aromatic urethane protecting group can be benzyloxycarbonyl, for example.

The aliphatic urethane protecting group can be, for example, tert-butoxycarbonyl or adamantyloxycarbonyl.

In accordance with the present invention, n can be an integer equal to or greater than 0. In some embodiments, n is no greater than 30, illustratively, n is 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0. In other embodiments, m is 0 or 1. In one embodiment, n=0. In another embodiment, n=0 when m=1.

In other embodiments, m=1, n=0, R is pyroglutamate, and X is 7-amino-4-methylcoumarin. For example, the compound can be characterized as pyro-glu-phe-lys-7-amido-4-methylcoumarin.

In other embodiments, the present invention provides a trifluoroacetyl derivative of the compound.

### II. Method

In another aspect, the present invention provides a method for determining an activity of a plasmin, the method comprising:

contacting the plasmin with a compound of the formula:

Rₘ-[Aₙ³A²A¹]-X (I)

wherein

A¹ is Lys; A² is Phe; A³ is an amino acid residue;

R is pyroglutamate or an N-terminal protecting group;

X is a fluorogenic group;

m is 0 or 1; and

n is an integer ≧0.

In one embodiment, m=1, n=0, R is pyroglutamate, and X is 7-amino-4-methylcoumarin. For example, the compound can be characterized as pyro-glu-phe-lys-7-amido-4-methylcoumarin.

The plasmin for which the activity is to be determined can be obtained from any source. In some embodiments, the plasmin is present in a biological fluid including, but not limited to, blood, serum, plasma, semen, and urine. In other embodiments, the plasmin is prepared by activating a plasminogen with a plasminogen activator such as, for example, streptokinase. For example, the plasminogen can be plasma-derived or recombinant plasminogen. In some embodiments, the plasmin is human plasmin. Plasmin from a non-human also is contemplated by the present invention including plasmin of a mouse, rat, monkey, cat, dog, horse, cow, etc.

In one embodiment, the method further comprises determining an amount of at least one reaction product produced by the reaction between the plasmin and the compound represented by the formula (I). For example, the amount of Rₘ-[Aₙ³A²A¹], X, or both can be determined, wherein the activity of the plasmin is determined on the basis of the amount of Rₘ-[Aₙ³A²A¹], X, or both.

In another embodiments, the method further comprises determining an amount of X produced by the reaction between the plasmin and the compound represented by the formula (I), wherein the activity of the plasmin is determined on the basis of the amount of X.

In other embodiments, the method further comprises determining the activity of the plasmin based on the difference in fluorescence between the X formed and the original compound.

In some aspects, the present invention provides a method for determining the pharmacokinetics of plasmin. For example, the method can provide for determining the presence and/or amount of administered plasmin at various physiological sites over time following administration. Pharmacokinetics can be evaluated by assessing levels of the administered plasmin. Methods can further comprise monitoring plasmin absorption and distribution, chemical modifications of the plasmin, and storage and elimination of the plasmin, and the like.

In one embodiment, the present invention provides a method of determining pharmacokinetic of a plasmin, the method comprising:

contacting the plasmin with a compound of the formula:

Rₘ-[Aₙ³A²A¹]-X (I)

wherein

A¹ is Lys; A² is Phe; A³ is an amino acid residue;

R is pyroglutamate or an N-terminal protecting group;

X is a fluorogenic group;

m is 0 or 1; and

n is an integer ≧0, wherein the plasmin is obtained from a biological sample from a subject administered with the plasmin. In one embodiment, m=1, n=0, R is pyroglutamate, X is 7-amino-4-methylcoumarin.

In other aspects, the present invention provides a method for determining a molecule that affects plasmin activity, the method comprising:

contacting the molecule with the plasmin in the presence of a compound of the formula:

Rₘ-[Aₙ³A²A¹]-X (I)

wherein

A¹ is Lys; A² is Phe; A³ is an amino acid residue;

R is pyroglutamate or an N-terminal protecting group;

X is a fluorogenic group;

m is 0 or 1; and

n is an integer ≧0. In one embodiment, m=1, n=0, R is pyroglutamate, X is 7-amino-4-methylcoumarin.

For example, in some embodiments, the method for determining the molecule can be a screening method for molecules that, directly or indirectly, affect plasmin activity. For example, the method can be a high-throughput screening for a molecule(s) that affect plasmin activity. The determined molecule can be any type of molecule that may affect plasmin activity. For example, the molecule to be determined can be a polypeptide, a compound referred to in the art as a small molecule, a chemical, bacteria, virus, etc.

### III. Kit

In another aspect, the present invention provides a kit comprising a compound. The compound is represented by the formula:

Rₘ-[Aₙ³A²A¹]-X (I)

wherein

A¹ is Lys; A² is Phe; A³ is an amino acid residue;

R is pyroglutamate or an N-terminal protecting group;

X is a fluorogenic group;

m is 0 or 1; and

n is an integer ≧0.

In one embodiment, m=1, n=0, R is pyroglutamate, X is 7-amino-4-methylcoumarin. In another embodiment, the compound is pyroglutamate-Phe-Lys-7-amido-4-methylcoumarin. In various embodiments, the components of the kit can be in separate compartments.

The following examples are given only to illustrate the present process and are not given to limit the invention. One skilled in the art will appreciate that the examples given only illustrate that which is claimed and that the present invention is only limited in scope by the appended claims.

### EXAMPLES

### Example 1

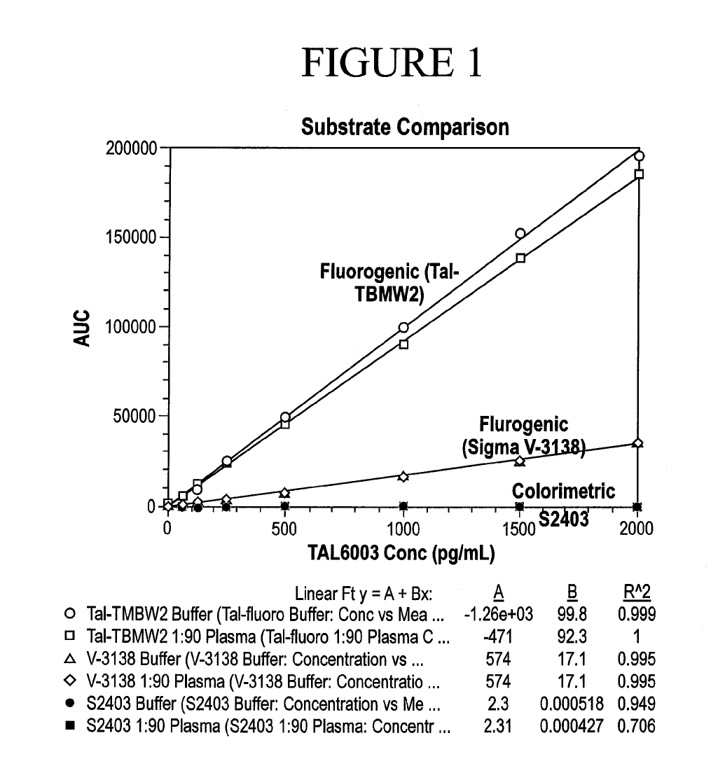

Plasmin hydrolysis of three different fluorogenic substrates to form peptide and the fluorogenic group 7-amido-4-methylcoumarin was determined. The three substrate were: (a) Tal-TBMW2, which corresponds to one embodiment of a compound of the present invention and which can be represented by the formula: pyroglutamate-Phe-Lys-7-amido-4-methylcoumarin; (b) Sigma V-3138 (Sigma Aldrich, St. Louis, Mo.); and (c) S2403 (Chromogenix, Milano, Italy). The results are shown in FIG. 1.

We claim:

1. A compound of the formula:
Rₘ-[Aₙ³A²A¹]-X (I)
wherein
A¹ is Lys; A² is Phe; A³ is an amino acid residue;
R is pyroglutamate or an N-terminal protecting group;
X is a fluorogenic group;
m is 0 or 1; and
n is an integer ≧0.

2. The compound of claim 1, wherein the fluorogenic group is selected from the group consisting of: 4-(substituted) coumarin, coumarin derivatives, hydroxy allyloxypropyl napthalimide quat, 4-methoxy-N-(3-N′N′-dimethylaminopropyl) napthalimide, 2-hydroxy-3-allyloxypropyl quat, 8-(3-vinylbenzyloxy)-1,3,6-pyrene trisulfonic acid, 8-(4-vinylbenzyloxy)-1,3,6-pyrene trisulfonic acid, 8-(allyloxy)-1,3,6-pyrene trisulfonic acid, 1-(substituted) naphthalene, 9-(substituted) anthracene, 2-(substituted) quinoline monohydrochloride, 2-(substituted) benzimidazole, 5-(substituted) fluorescein, 3-(substituted)-6,7-dimethoxy-1-methyl-2(1H)-quinoxazolinone, mixtures thereof and derivatives thereof.

3. The compound of claim 1, wherein the fluorogenic group is 7-amino-4-methylcoumarin.

4. The compound of claim 1, wherein R is pyroglutamate.

5. The compound of claim 1, wherein the N-terminal protecting group is selected from the group consisting of: an acyl protecting group, an aromatic urethane protecting group, and an aliphatic urethane protecting group.

6. The compound of claim 1, wherein the N-terminal protecting group is succinyl or methoxysuccinyl.

7. The compound of claim 5, wherein the acyl protecting group is selected from the group consisting of: succinyl, methoxysuccinyl, acetyl, benzoyl, and trifluoroacetyl.

8. The compound of claim 5, wherein the aromatic urethane protecting group is benzyloxycarbonyl.

9. The compound of claim 5, wherein the aliphatic urethane protecting group is tert-butoxycarbonyl or adamantyloxycarbonyl.

10. The compound of claim 1, wherein n=0.

11. The compound of claim 1, wherein m=1, wherein, n=0, wherein R is pyroglutamate, wherein X is 7-amino-4-methylcoumarin.

12. A trifluoroacetyl derivative of the compound of claim 1.

13. A method for determining an activity of a plasmin, the method comprising:
contacting the plasmin with a compound of the formula:
Rₘ-[Aₙ³A²A¹]-X (I)
wherein
A¹ is Lys; A² is Phe; A³ is an amino acid residue;
R is pyroglutamate or an N-terminal protecting group;
X is a fluorogenic group;
m is 0 or 1; and
n is an integer ≧0.

14. The method of claim 13, wherein n=0, wherein R is pyroglutamate, wherein X is 7-amino-4-methylcoumarin.

15. The method of claim 13, wherein the plasmin is prepared from a plasma-derived plasminogen or a recombinant plasminogen.

16. The method of claim 13 further comprising determining an amount of at least one of R-[Aₙ³A²A¹] and X produced by the reaction between the plasmin and the compound represented by the formula (I), wherein the activity of the plasmin is determined on the basis of the amount of at least R-[Aₙ³A²A¹], X, or both.

17. The method of claim 13 further comprising determining an amount of X produced by the reaction between the plasmin and the compound represented by the formula (I), wherein the activity of the plasmin is determined on the basis of the amount of X.

18. The method of claim 13 further comprising determining the activity of the plasmin based on the difference in fluorescence between the X formed and the original compound.

19. A method of determining pharmacokinetic of a plasmin, the method comprising:
contacting the plasmin with a compound of the formula:
Rₘ-[Aₙ³A²A¹]-X (I)
wherein
A¹ is Lys; A² is Phe; A³ is an amino acid residue;
R is pyroglutamate or an N-terminal protecting group;
X is a fluorogenic group;
m is 0 or 1; and
n is an integer ≧0, wherein the plasmin is obtained from a biological sample from a subject administered with the plasmin.

20. A method for determining a molecule that affects plasmin activity, the method comprising:
contacting the molecule with the plasmin in the presence of a compound of the formula:
Rₘ-[Aₙ³A²A¹]-X (I)
wherein
A¹ is Lys; A² is Phe; A³ is an amino acid residue;
R is pyroglutamate or an N-terminal protecting group;
X is a fluorogenic group;
m is 0 or 1; and
n is an integer ≧0.
